(19) United States
(12) Patent Application Publication
(10) Pub. No.: US 2024/0360478 A1
(43) Pub. Date: Oct. 31, 2024

(54) Efficiency of viral delivery to cell lines differentiated from induced pluripotent stem cells

(71) Applicant: The Regents of the University of California, Oakland, CA (US)

(72) Inventors: Shawn L. Shafer, San Francisco, CA (US); Rustam Esanov, San Francisco, CA (US); Laralynne Przybyla, San Francisco, CA (US)

(73) Assignee: The Regents of the University of California, Oakland, CA (US)

(21) Appl. No.: 18/765,171

(22) Filed: Jul. 5, 2024

Related U.S. Application Data
(63) Continuation of application No. PCT/US23/61114, filed on Jan. 23, 2023.
(60) Provisional application No. 63/304,614, filed on Jan. 30, 2022.

(51) Int. Cl. C12N 15/90; C12N 9/22; C12N 15/11; C12N 15/86

(52) U.S. Cl. CPC C12N 15/907; C12N 9/22; C12N 15/11; C12N 15/86; C12Y 301/05

(57) ABSTRACT

Methods and compositions for genetically altering induced pluripotent stem cells (iPSCs) to allow for efficient viral delivery to differentiated cell lines derived from this background.

### REFERENCE TO A SEQUENCE LISTING

A Sequence Listing in XML format is incorporated by reference into the specification. The name of the XML file containing the Sequence Listing is SF22-096-2US.xml. The XML file is 6,623 bytes and was created Jul. 4, 2024.

### INTRODUCTION

Recombinant viruses, and particularly lentivirus, are ubiquitous tools in functional genomics research because of their ability to target and enter mammalian cells and deliver a user-defined genetic payload, such as CRISPR constructs, reporters, gene expression cassettes, etc., to these cells at high efficiencies. In parallel, the field is moving from studying immortalized cancer lines towards diploid iPSC-derived models in the belief that these cell lines are more representative of actual human biology. However, because many iPSC-derived models are resistant to viral delivery, researchers must work around this issue using suboptimal approaches, which primarily consist of a) delivering the viral payload to the undifferentiated iPSC line or b) creating inducible constructs that can be activated post-iPSC differentiation. Both of these approaches have significant drawbacks: for viral delivery to undifferentiated iPSCs, genetically active payloads (gene expression, CRISPR, etc) may interfere with the differentiation process. This approach also does not get around the “bottleneck” effect of iPSC-differentiation, where only a portion of the parental iPSCs give rise to the differentiated population, skewing statistical screening approaches. Inducible constructs (b) get around these considerations, but are hampered by the unpredictable effects of transgene silencing post-differentiation.

### SUMMARY OF THE INVENTION

The invention provides methods and compositions for genetically altering induced pluripotent stem cells (iPSCs) to allow for efficient viral delivery to differentiated cell lines derived from this background, particularly wherein the genetic alteration comprises a genetically disrupted SAMHD1 (SAM and HD Domain Containing Deoxynucleoside Triphosphate Triphosphohydrolase 1) gene.

In an aspect the invention provides a method for improving the efficiency of viral delivery, the method comprising transforming with recombinant virus, a cell differentiated from an induced pluripotent stem cell (iPSCs) comprising a genetically disrupted SAMHD1 gene.

In an aspect the invention provides an induced pluripotent stem cell (iPSC) comprising a genetically disrupted SAMHD1 gene, i.e the stem cell prior to being differentiated and/or transformed.

In an aspect the invention provides a cell transformed with a recombinant virus, wherein the cell is differentiated from an induced pluripotent stem cell (iPSC) comprising a genetically disrupted SAMHD1 gene.

In an aspect the invention provides a gRNA for targeted disruption of SAMHD1, and/or an editing (e.g. CRISPR) construct comprising such gRNA; exemplary such gRNAs below.

In an aspect the invention provides a therapeutic process like cell engineering for ex vivo cellular therapies, where the therapeutic cargo is delivered via a viral vector, employing the improvements disclosed herein.

In an aspect, the invention provides methods and compositions for genetically disrupting the SAMHD1 gene to enhance lentiviral delivery to iPSC-derived cells, essentially as disclosed herein.

In embodiments:
the SAMHD1 comprises the sequence of human SAMHD1, UniProtKB-Q9Y3Z3 (SAMH1_HUMAN), or a sequence having a 90+% sequence identity thereto;
the gRNA comprises a sequence of gRNA1, gRNA2 or gRNA3, herein, or a sequence having a 90+% sequence identity thereto;
the method provides an improvement that comprises an increase in the proportion of transformed cells by at least 20 or 50% compared to cells with a non-disrupted SAMHD1 gene, or at least 20 or 50% decrease in cell death, or at least as demonstrated herein (e.g. FIGS. 2B, 2C, etc);
the SAMHD1 genetic disruption is effected by a loss-of-function knockout, knock-down, mutation or edit sufficient to effect improved efficiency of viral delivery, and is effected by recombination knockout, TALEN and CRISPR/Cas genome editing systems, zinc finger nucleases, etc;
the stem cell is differentiated, and/or the stem cell comprises a gRNA-mediated genetic knockout of the SAMHD1 gene;
the differentiated cell is selected from macrophages, microglia, hematopoietic progenitor cells (HPCs) and natural killer (NK) cells; and/or
the recombinant virus is selected from lentivirus, poxvirus, adenovirus, adeno-associated virus, retrovirus, human foamy virus (HFV) and herpes virus.

The invention encompasses all combinations of the particular embodiments recited herein, as if each combination had been laboriously recited.

### BRIEF DESCRIPTION OF THE DRAWINGS

FIG. 1A-B. SAMHD1 knockout strategy and confirmation in human iPSCs. Three gRNAs targeting exon 1 of SAMHD1 (SEQ ID NO:1) (A) were selected and delivered with Cas9 via electroporation. PCR confirming SAMHD1 knockout (B).

FIG. 2A-C. SAMHD1 knockout at iPSC stage increases lentiviral transduction efficiency in differentiated macrophages. Wild-type and SAMHD1 knockout (KO) iPSCs were differentiated into macrophages and transduced with lentivirus expressing GFP cassette. Fluorescence was assessed via fluorescent microscopy (A) and flow cytometry (B). Quantification of flow cytometry data from two independent experiments shown in C.

FIG. 3A-C. Transcriptional and cytokine profiles of macrophages differentiated from iPSCs with SAMHD1 knockout are nearly identical to wild-type. Volcano plot from RNA-seq analysis of WT and SAMHD1 KO macrophages (A), significant differentially expressed genes labeled in red. Cytokine profiles of macrophages at basal state (B) and activated state after LPS induction (C).

FIG. 4A-C. Lentiviral transduction efficiency in iPSC-derived macrophages (iMacrophage) from WT and SAMHD1 KO lines. Percent BFP and GFP positive cells 72 h after transduction using CD55i (A), CRISPRi (B) and commercial (C) lentiviral batches. Transduction efficiency was assessed by looking at percentage of BFP (A,B) and GFP (C) positive cells via flow cytometry analysis. Experiment was performed in triplicates; graphs show average percentages and error bars represent standard deviation values.

FIG. 5A-F. Lentiviral transduction efficiency in iPSC-derived microglia from WT and SAMHD1 KO lines. Microglia were differentiated using two independent protocols: a commercially available kit from StemCell Technologies (StemCell Tech iMicroglia, A-D) and an in-house hypoxia microglia differentiation protocol (Hypoxia iMicroglia, E-F). Transduction efficiency is shown as percent BFP and GFP positive cells 72 hours post transduction. For StemCell Tech iMicroglia, 4 lentiviral batches were used: CD55i (A), CRISPRi (B), commercial from Vectorbuilder (C) and commercial from Sigma (D). For Hypoxia iMicroglia, two lentiviral batches were used: CD55i (E) and commercial from Sigma (F). Experiment was performed in triplicates; graphs show average percentages and error bars represent standard deviation values.

FIG. 6A-B. Lentiviral transduction efficiency in iPSC-derived NK cells and neurons from SAMHD1 KO and WT lines. Cells were incubated in the presence of CD55i lentivirus overnight and percentage of cells expressing BFP was measured using flow cytometry 72 h after transduction. Experiment was performed once in iNK cells (A) and four times in iNeurons (B), average values from the replicates are shown, error bars represent standard deviation.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

Inactivation or knocking out the SAMHD1 gene at the iPSC stage substantially increases the efficiency of lentiviral delivery to the resulting differentiated cells, vastly increasing the potential for robust and direct functional genomics screening in those models.

This invention enables researchers to perform functional genomics screens directly in iPSC-derived lineages, such as macrophages, NK cells, and neurons, in a way that is not currently possible, greatly enhancing the ability to interrogate the biology of these cells in normal and disease backgrounds. Genetically inactivating the SAMHD1 gene in iPSCs allows for viral delivery directly to the differentiated cells without substantially altering the core biology of the derived cell type, resolving the drawbacks of the current approaches described above. The genetically engineered iPS cells described herein can be used to derive differentiated cell lines for functional genomics screening or therapeutic applications.

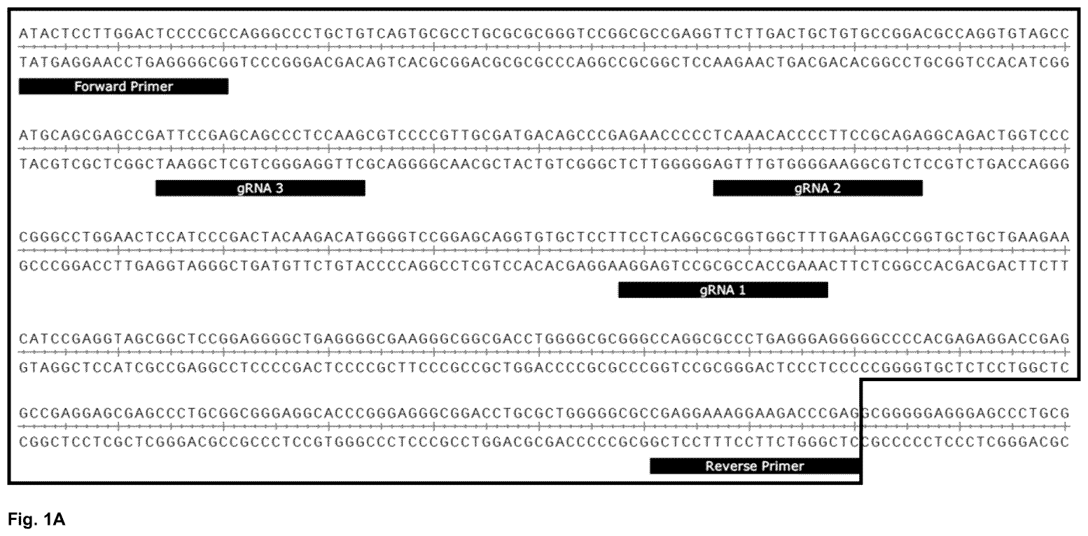

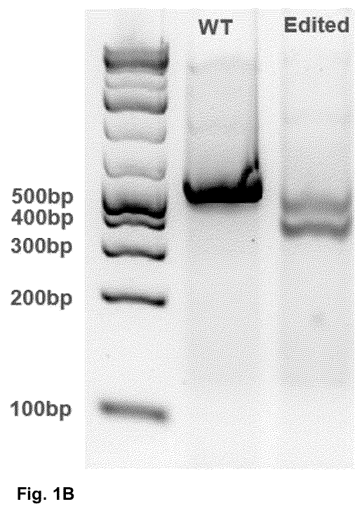

A description of the SAMHD1 knockout strategy and confirmation in human iPSCs are shown in FIG. 1. Three gRNAs targeting exon 1 of SAMHD1 (FIG. 1A) were selected and delivered with Cas9 via electroporation. PCR confirming SAMHD1 knockout (FIG. 1B).

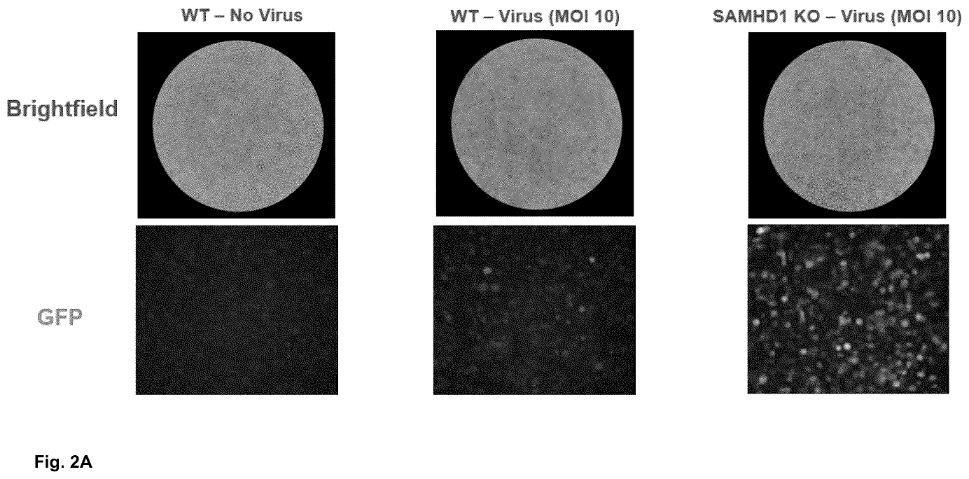

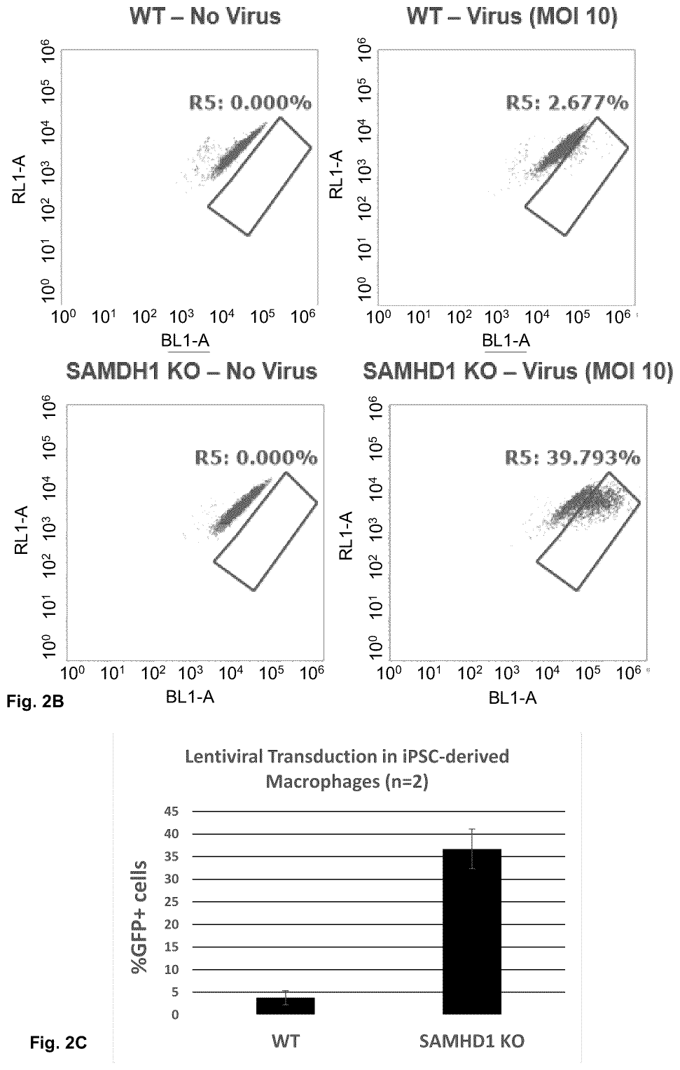

SAMHD1 knockout at iPSC stage increases lentiviral transduction efficiency in differentiated macrophages; see, FIG. 2. Wild-type and SAMHD1 knockout (KO) iPSCs were differentiated into macrophages and transduced with lentivirus expressing GFP cassette. Fluorescence was assessed via fluorescent microscopy (FIG. 2A) and flow cytometry (FIG. 2B). Quantification of flow cytometry data from two independent experiments is shown in FIG. 2C.

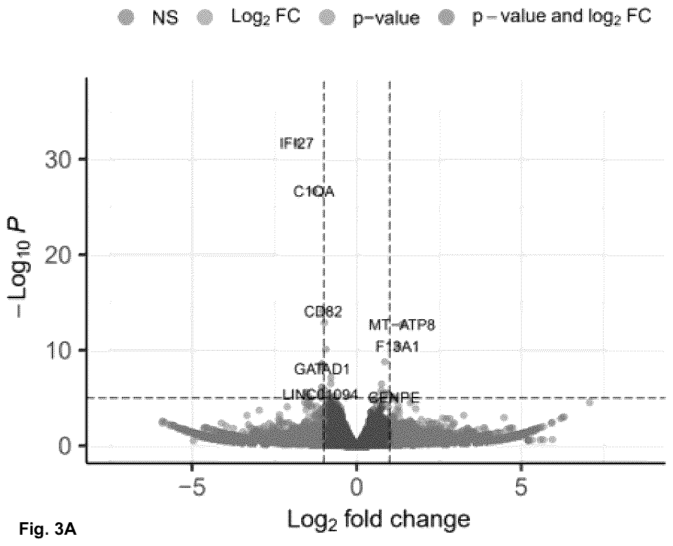

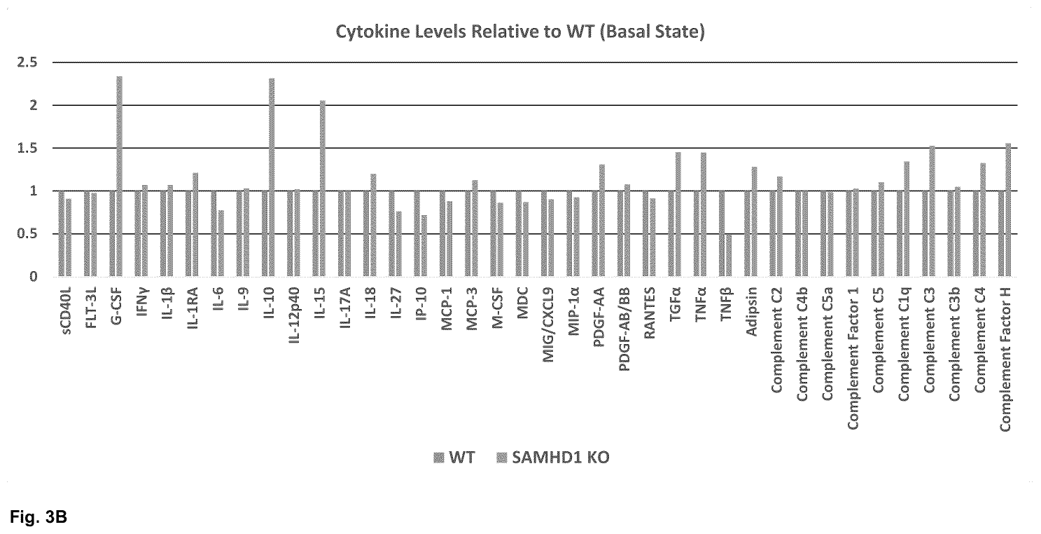

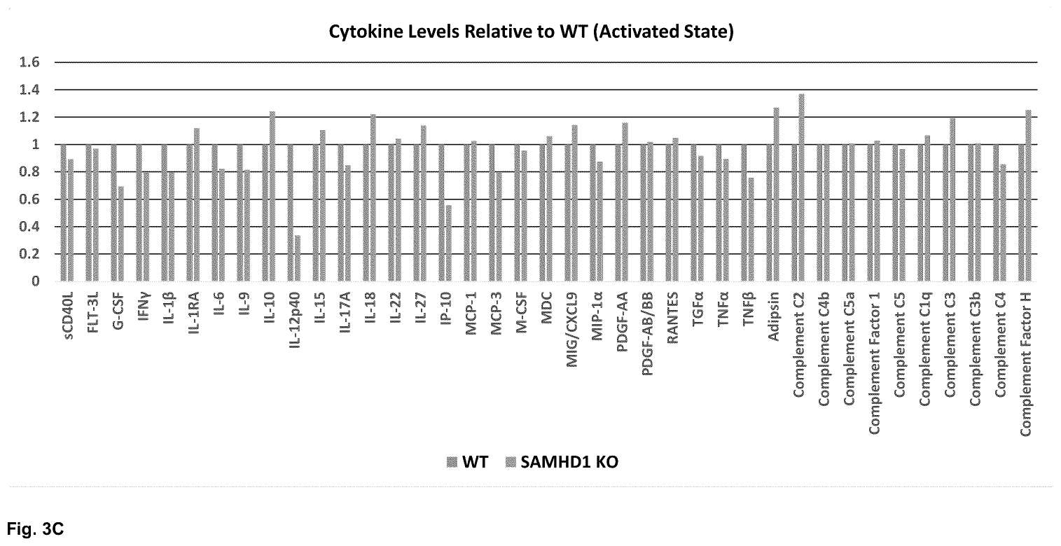

Transcriptional and cytokine profiles of macrophages differentiated from iPSCs with SAMHD1 knockout are nearly identical to wild-type; see, FIG. 3. Volcano plot from RNA-seq analysis of WT and SAMHD1 KO macrophages (FIG. 3A), wherein significant differentially expressed genes are labeled in red. Cytokine profiles of macrophages at basal state (B) and activated state after LPS induction (C).

```
gRNAs targeting exon 1 of SAMHD1:
gRNA1:
(SEQ ID NO: 2)
AAAGCCACCGCGCCUGAGGA
gRNA2:
(SEQ ID NO: 3)
UCUGCGGAAGGGGUGUUUGA
gRNA3:
(SEQ ID NO: 4)
CUUGGAGGGCUGCUCGGAAU
UniProtKB - Q9Y3Z3 (SAMH1_HUMAN)
(SEQ ID NO: 5)
                10 20 30 40
MQRADSEQPS KRPRCDDSPR TPSNTPSAEA DWSPGLELHP
                50 60 70 80
DYKTWGPEQV CSFLRRGGFE EPVLLKNIRE NEITGALLPC
                90 100 110 120
LDESRFENLG VSSLGERKKL LSYIQRLVQI HVDTMKVIND
              130 140 150 160
PIHGHIELHP LLVRIIDTPQ FQRLRYIKQL GGGYYVFPGA
              170 180 190 200
SHNRFEHSLG VGYLAGCLVH ALGEKQPELQ ISERDVLCVQ
              210 220 230 240
IAGLCHDLGH GPFSHMEDGR FIPLARPEVK WTHEQGSVMM
              250 260 270 280
FEHLINSNGI KPVMEQYGLI PEEDICFIKE QIVGPLESPV
              290 300 310 320
EDSLWPYKGR PENKSFLYEI VSNKRNGIDV DKWDYFARDC
              330 340 350 360
HHLGIQNNFD YKRFIKFARV CEVDNELRIC ARDKEVGNLY
              370 380 390 400
DMFHTRNSLH RRAYQHKVGN IIDTMITDAF LKADDYIEIT
              410 420 430 440
GAGGKKYRIS TAIDDMEAYT KLIDNIFLEI LYSTDPKLKD
              450 460 470 480
AREILKQIEY RNLFKYVGET QPTGQIKIKR EDYESLPKEV
              490 500 510 520
ASAKPKVLLD VKLKAEDFIV DVINMDYGMQ EKNPIDHVSF
              530 540 550 560
YCKTAPNRAI RITKNQVSQL LPEKFAEQLI RVYCKKVDRK
              570 580 590 600
SLYAARQYFV QWCADRNFTK PQDGDVIAPL ITPQKKEWND
              610 620
STSVQNPTRL REASKSRVQL FKDDPM
```

### Example 1. Effect of SAMHD1 Knockout on Transduction Efficiency in Various iPSC-Derived Cell Types

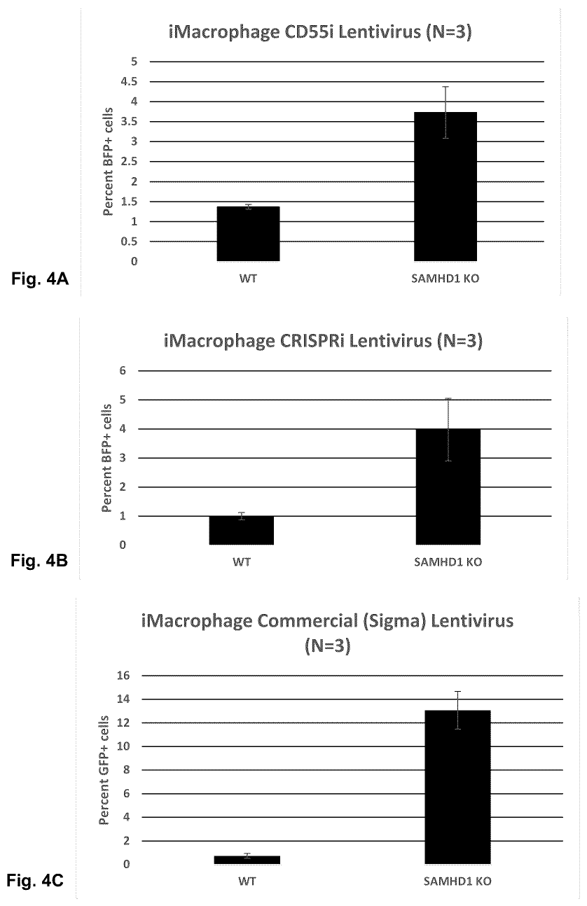

In this example iPSC-derived macrophages from SAMHD1 KO line were confirmed to demonstrate an increase in transduction efficiency compared to WT controls (FIG. 4A-C). Three different lentiviral constructs were utilized that express a fluorescent protein: CD55i lentivirus (prepared in-house, expressing BFP), CRISPRi lentivirus (prepared in-house, expressing BFP) and a commercial lentivirus (Sigma-Aldrich, expressing GFP, Cat #17-10387). Cells were incubated in the presence of the lentivirus overnight and expression of fluorescent protein was assessed using flow cytometry after 72 hours. These results indicate an increase in transduction efficiency in SAMHD1 KO macrophages for all three lentiviral batches.

### Example 2. SAMHD1 KO Increases Lentiviral Transduction in Another Myeloid Cell Type: Microglia

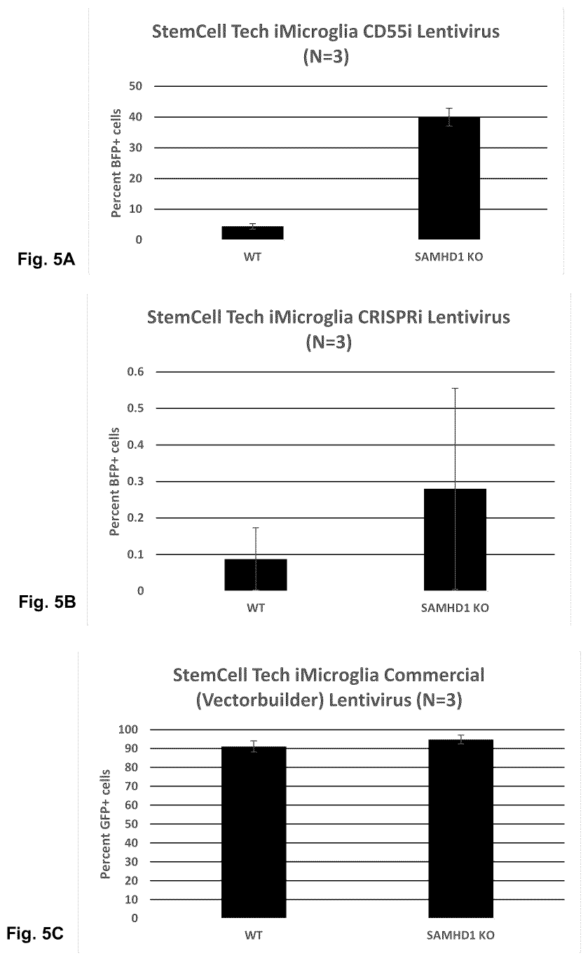

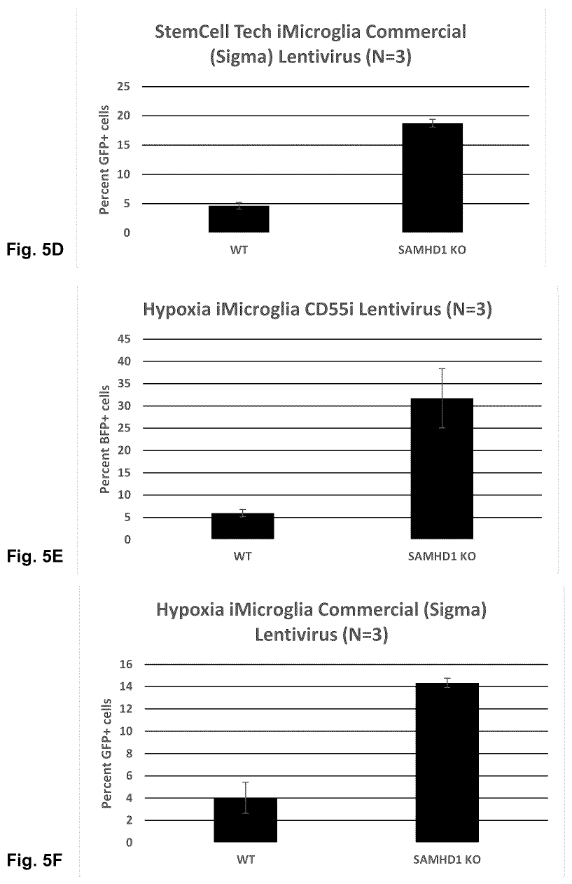

iPSC-derived microglia from WT and SAMHD1 KO lines were generated using two independent protocols: StemCell Technologies (StemCell Tech iMicroglia, Cat #100-0019) and an in-house protocol using hypoxia-based differentiation (Hypoxia iMicroglia). After differentiation, cells were incubated in the presence of various lentiviral batches overnight and expression of fluorescent proteins was assessed using flow cytometry 72 h post transduction (FIG. 5A-F). Overall, transduction was increased in SAMHD1 KO microglia compared to WT control, with the exception of CRIPSRi and commercial (Vectorbuilder) lentiviral batches in StemCell Tech iMicroglia.

### Example 3. An Evaluation of Lentiviral Transduction Efficiency in Natural Killer Cells (iNK) and Neurons (iNeurons) Derived from WT and SAMHD1 KO iPSC Lines

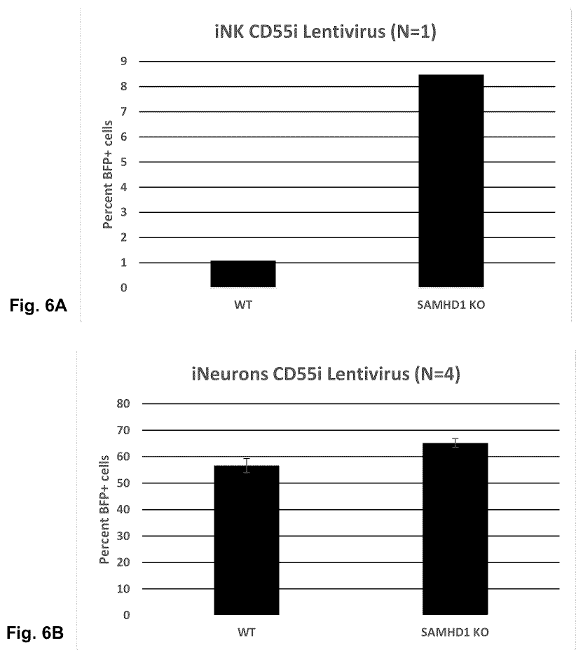

Cell types were differentiated using a commercially available kit from StemCell Technologies (Cat #100-0170 and Cat #08600). Transduction efficiency was increased approximately 8-fold in iNK cells from SAMHD1 KO line whereas in iNeurons only a modest transduction efficiency increase was observed (FIG. 6A-B).

1. An induced pluripotent stem cell comprising a genetically disrupted SAMHD1 (SAM and HD Domain Containing Deoxynucleoside Triphosphate Triphosphohydrolase 1) gene.

2. A cell transformed with a recombinant virus, wherein the cell is differentiated from an induced pluripotent stem cell of claim 1.

3. A method for genetically altering an induced pluripotent stem cell for efficient viral delivery to differentiated cell lines derived therefrom, comprising genetically disrupting a SAMHD1 gene in the stem cell.

4. A method of viral delivery to a cell of claim 1, the method comprising transforming with recombinant virus a cell differentiated from an induced pluripotent stem cell comprising a genetically disrupted SAMHD1 gene.

5. A composition for making a cell of claim 1, comprising a gRNA configured for targeted disruption of SAMHD1, and/or an editing (e.g. CRISPR) construct configured for targeted disruption of SAMHD1.

6. A method of cell engineering for ex vivo cellular therapies, comprising delivering to a target cell a therapeutic cargo via a viral vector, wherein the cell is differentiated from an induced pluripotent stem cell comprising a genetically disrupted SAMHD1 gene.

7. The method of claim 6, further comprising differentiating the target cell from the induced pluripotent stem cell.

8. The method of claim 6, further comprising genetically disrupting the SAMHD1 gene to generate the induced pluripotent stem cell, and differentiating the target cell from the induced pluripotent stem cell.

9. The method of claim 6, further comprising genetically disrupting the SAMHD1 gene to generate the induced pluripotent stem cell, and differentiating the target cell from the induced pluripotent stem cell, wherein the genetic disruption is effected by a gRNA configured for targeted disruption of SAMHD1.

10. The method of claim 6, further comprising genetically disrupting the SAMHD1 gene to generate the induced pluripotent stem cell, and differentiating the target cell from the induced pluripotent stem cell, wherein the genetic disruption is effected by a gRNA configured for targeted disruption of SAMHD1, wherein the gRNA comprises a sequence of:
gRNA1:
(SEQ ID NO: 2)
AAAGCCACCGCGCCUGAGGA,
gRNA2:
(SEQ ID NO: 3)
UCUGCGGAAGGGGUGUUUGA,
or
gRNA3:
(SEQ ID NO: 4)
CUUGGAGGGCUGCUCGGAAU,
or a sequence having a 90+% sequence identity thereto.

11. The method of claim 6, further comprising genetically disrupting the SAMHD1 gene to generate the induced pluripotent stem cell, and differentiating the target cell from the induced pluripotent stem cell, wherein the genetic disruption is effected by a gene editing construct configured for targeted disruption of SAMHD1.

12. The method of claim 6, further comprising genetically disrupting the SAMHD1 gene to generate the induced pluripotent stem cell, and differentiating the target cell from the induced pluripotent stem cell, wherein the genetic disruption is effected by a loss-of-function knockout or knock-down sufficient to effect improved efficiency of viral delivery.

13. The method of claim 6, wherein the SAMHD1 comprises the sequence of human SAMHD1, UniProtKB-Q9Y3Z3 (SAMH1_HUMAN), or a sequence having a 90+% sequence identity thereto.

14. The method of claim 6, wherein the SAMHD1 disruption increases lentiviral transduction efficiency in differentiated macrophages by at least 50% compared to comparable cells with a non-disrupted SAMHD1 gene, as assessed by flow cytometry.

15. The method of claim 6, wherein the differentiated cell is a macrophage cell.

16. The method of claim 6, wherein the differentiated cell is a microglia cell.

17. The method of claim 6, wherein the differentiated cell is a hematopoietic progenitor cell. (HPC).

18. The method of claim 6, wherein the differentiated cell is a natural killer (NK) cell.

19. The method of claim 6, wherein the recombinant virus is selected from lentivirus, pox virus, adenovirus, adeno-associated virus, retrovirus, human foamy virus (HFV) and herpes virus.

20. The method of claim 6, wherein the recombinant virus is lentivirus.


SEQUENCE LISTING (5 sequences)

SEQ ID NO: 1 - DNA, 499 bp, Homo sapiens
  source 1..499: mol_type = genomic DNA
atactccttggactccccgccagggccctgctgtcagtgcgcctgcgcgcgggtccggcgccgaggttcttgactgctgtgccggacgccaggtgtagccatgcagcgagccgattccgagcagccctccaagcgtccccgttgcgatgacagcccgagaaccccctcaaacaccccttccgcagaggcagactggtccccgggcctggaactccatcccgactacaagacatggggtccggagcaggtgtgctccttcctcaggcgcggtggctttgaagagccggtgctgctgaagaacatccgaggtagcggctccggaggggctgaggggcgaagggcggcgacctggggcgcgggccaggcgccctgagggagggggccccacgagaggaccgaggccgaggagcgagccctgcggcgggaggcacccgggagggcggacctgcgctgggggcgccgaggaaaggaagacccgagcgggggagggagccctgcg

SEQ ID NO: 2 - RNA, 20 nt, Homo sapiens
  source 1..20: mol_type = other RNA
aaagccaccgcgcctgagga

SEQ ID NO: 3 - RNA, 20 nt, Homo sapiens
  source 1..20: mol_type = other RNA
tctgcggaaggggtgtttga

SEQ ID NO: 4 - RNA, 20 nt, Homo sapiens
  source 1..20: mol_type = other RNA
cttggagggctgctcggaat

SEQ ID NO: 5 - protein, 626 aa, Homo sapiens
MQRADSEQPSKRPRCDDSPRTPSNTPSAEADWSPGLELHPDYKTWGPEQVCSFLRRGGFEEPVLLKNIRENEITGALLPCLDESRFENLGVSSLGERKKLLSYIQRLVQIHVDTMKVINDPIHGHIELHPLLVRIIDTPQFQRLRYIKQLGGGYYVFPGASHNRFEHSLGVGYLAGCLVHALGEKQPELQISERDVLCVQIAGLCHDLGHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPKLKDAREILKQIEYRNLFKYVGETQPTGQIKIKREDYESLPKEVASAKPKVLLDVKLKAEDFIVDVINMDYGMQEKNPIDHVSFYCKTAPNRAIRITKNQVSQLLPEKFAEQLIRVYCKKVDRKSLYAARQYFVQWCADRNFTKPQDGDVIAPLITPQKKEWNDSTSVQNPTRLREASKSRVQLFKDDPM